(19) United States
(12) Patent Application Publication
(10) Pub. No.: US 2024/0000559 A1
(43) Pub. Date: Jan. 4, 2024

(54) COMPUTATIONAL PLANNING OF SURGICAL RECONSTRUCTION

(71) Applicant: Children's Medical Center Corporation, Boston, MA (US)

(72) Inventors: David Hoganson, Chestnut Hill, MA (US); Peter E. Hammer, Needham, MA (US); Emily Eickhoff, Brookline, MA (US); Noah Schulz, Cambridge, MA (US)

(73) Assignee: Children's Medical Center Corporation, Boston, MA (US)

(21) Appl. No.: 18/030,467

(22) PCT Filed: Oct. 7, 2021
(86) PCT No.: PCT/US2021/053977

§ 371 Date: Apr. 5, 2023

Related U.S. Application Data
(60) Provisional application No. 63/088,638, filed on Oct. 7, 2020.

(51) Int. Cl. A61F 2/06; A61B 34/10; B33Y 10/00; B33Y 50/00; B33Y 80/00

(52) U.S. Cl. CPC A61F 2/062; A61B 34/10; B33Y 10/00; B33Y 50/00; B33Y 80/00; A61B 2034/105; A61B 2034/102; A61F 2240/002; A61B 2034/108

(57) ABSTRACT

Methods and systems for cardiovascular structure reconstruction are provided. A method of reconstructing a cardiovascular structure may include imaging the cardiovascular structure, producing a three-dimensional model of the cardiovascular structure based on the imaging, and creating a three-dimensional model of a patch corresponding to the three-dimensional model of the cardiovascular structure, where the patch is configured to reconstruct the cardiovascular structure to a normal geometry. A patch for a cardiovascular structure may include at least one layer of anisotropic material including an outermost perimeter and one or more notches extending inward from the outermost perimeter, where each of the one or more notches creates a discontinuity in the outermost perimeter. In some cases, each of the one or more notches includes a first edge and a second opposing edge, and the patch further includes one or more sutures configured to secure the first edge to the second opposing edge.

### RELATED APPLICATIONS

This application claims priority to and the benefit under 35 U.S.C. § 119(e) of U.S. Provisional App. No. 63/088,638, filed Oct. 7, 2020, the disclosure is which is herein incorporated by reference in its entirety.

### FIELD

Disclosed embodiments are related to computational planning of surgical reconstruction, related systems, and related methods of use of such systems.

### BACKGROUND

Surgical reconstructions have typically been performed by visual assessment of the surgeon in the planning, shaping, and implantation of patches and other reconstructive materials to achieve the desired final anatomy of the reconstruction. These approaches have typically been heavily weighted towards visual estimation. The utilization of a visually based estimation system and post-reconstruction assessment does leave maximum flexibility for the surgeon to adjust the reconstruction based upon the eye of the particular pathology, but it also leaves room for a tremendous variability in the geometric outcome of the reconstruction. Although these concepts apply across most surgical disciplines, cardiovascular reconstructions are typically performed this way. Both the shape and diameter of cardiovascular reconstructions are important to achieve appropriate hemodynamics within the cardiovascular system after the reconstruction. This applies to intracardiac reconstruction as well as vascular reconstructions involving the systemic arterial, pulmonary arterial, pulmonary venous, and systemic venous reconstructions.

### SUMMARY

In some embodiments, a method of reconstructing a cardiovascular structure includes obtaining a three-dimensional model of the cardiovascular structure based on information regarding the cardiovascular structure, and creating a three-dimensional model of a patch corresponding to the three-dimensional model of the cardiovascular structure, wherein the patch is configured to reconstruct the cardiovascular structure to a normal geometry.

In some embodiments, a patch for a cardiovascular structure includes at least one layer of anisotropic material including an outermost perimeter, and one or more cutouts formed in the at least one layer of anisotropic material.

In some embodiments, a method of forming a patch for a cardiovascular structure includes projecting a two-dimensional plan of the patch onto at least one layer of anisotropic material, wherein the two-dimensional plan is a flattened three-dimensional model of the patch, wherein the two-dimensional plan of the patch includes an outermost perimeter and one or more cutouts in the patch.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1C depict some examples of an aortic arch with various conditions;

FIGS. 2A-2C depict some examples of an aortic arch after reconstruction according to conventional methods;

FIGS. 3A-3D depict an exemplary conventional method for aortic arch reconstruction;

FIGS. 4A-4E depict one embodiment of a method of reconstructing an aorta to a normal geometry;

FIGS. 5A-5C depict one embodiment of a method of flattening a modeled three-dimensional patch;

FIG. 6 depicts a schematic of one embodiment of a biaxial testing of anisotropic patch material

FIG. 7 depicts exemplary graphs of test data of from biaxial testing of anisotropic patch material according to exemplary embodiments described herein;

FIG. 8 depicts an exemplary graph of test data of from biaxial testing of pulmonary homograft patch material according to exemplary embodiments described herein;

FIGS. 9A-9F depict one embodiment of a method of reconstructing an aorta to a normal geometry;

FIGS. 10A-10E depict one embodiment of a method of molding an aorta for validating the methods and systems described herein;

FIG. 11 depicts a workflow schematic for one embodiment of a method of reconstructing an aorta to a normal geometry; and

FIGS. 12A-12G depicts one embodiment of a method of reconstructing a cardiovascular structure.

### DETAILED DESCRIPTION

In some cases, imprecision in cardiovascular reconstruction can lead to residual narrowing or an oversized area within the heart or blood vessels. These anatomic reconstructions are often subject to growth in remodeling and imprecise reconstruction can often hamper growth of the geometric reconstruction if the initial operation was not ideal. Areas of reconstruction with residual narrowing or larger than normal diameters can cause important energy loss within the cardiovascular system they can have devastating long-term consequences including potentially needing additional procedures or resulting in an increased risk of cardiovascular complications such as a stroke and myocardial infarction. For example, reconstruction of the aortic arch is a common procedure in pediatric cardiac surgery. Residual narrowing of the aortic arch can significantly increase the workload of the left ventricle. This impacts only the left ventricle long-term with thickening and can lead to ventricular dysfunction. It also increases the risk of early myocardial infarction and stroke in these patients in early adulthood. Conversely, the area of dilation of the transverse arch with a taper down to the descending aorta also leads to energy loss and elevated complex impedance, which also increases the afterload on the left ventricle. A similar clinical scenario exists for pulmonary artery reconstructions where achieving main and branch pulmonary artery size and shape consistent with normal for the size of the child is imperative. Another clinical scenario is in patients where the reconstruction of the intracardiac anatomy including but not limited to connecting the left ventricle to the aorta. In such instances, often a complex baffle is constructed within the heart to achieve reconstruction. Areas of narrowing of this baffle either acutely or as the child grows can substantially increase the workload of the left ventricle to get blood out of the heart. This could lead to reoperation or compromise of the left ventricle function.

In view of the above, the inventors have appreciated methods and systems that address the conventional challenges associated with reconstruction of the aorta, pulmonary arteries and other cardiovascular structures during surgery. The inventors have appreciated the benefits of a method that includes characterizing (e.g., imaging) a pulmonary structure, determining stress strain curves for a flat patch applied to the pulmonary structure, and determining one or more portions of the patch to remove such that the patch may be pre-curved prior to installation on the pulmonary structure. The inventors have also appreciated the benefits of a patch for a pulmonary structure that is pre-curved before application to the pulmonary structure. Such a patch may provide support for the pulmonary structure in accordance with the stress and strain limits of the patch, and thereby yield a more consistent surgical outcome when used in surgical methods according to exemplary embodiments described herein. Exemplary embodiments of methods and system having such benefits are discussed further below. Additionally, other features and advantages of the invention will be apparent from the following detailed description.

In some embodiments, a method of reconstructing a cardiovascular structure may include characterizing the cardiovascular structure. In some embodiments, characterizing the cardiovascular structure may include producing one or more images of the cardiovascular structure. For example, in some embodiments, imaging the cardiovascular structure may include using magnetic resonance imaging (MRI), computerized tomography (CT), ultrasound, or another suitable imaging process. The method may also include producing a three-dimensional model of the cardiovascular structure based on the one or more images. For example, in some embodiments, the one or more images may be sent to a processor (e.g., via a wired or wireless communication protocol) which is configured to process the one or more images into a three-dimensional computer model of the cardiovascular structure. In some embodiments, a doctor (e.g., a surgeon), medical engineer or other medical professional may create the three-dimensional model with the assistance of computer aided design (CAD) software (e.g., at a user interface). In some embodiments, the method may also include creating a three-dimensional model of a patch corresponding to the three-dimensional model of the cardiovascular structure, where the patch is configured to reconstruct the cardiovascular structure to a normal geometry. In some embodiments, the three-dimensional model of the patch may be based at least in part in a stress strain curve of an anisotropic material of the patch. That is, the three-dimensional model of the patch may be configured to achieve a normal geometry of the cardiovascular structure when accounting for the deformation and strength of the patch. In some embodiments, the three-dimensional model of the patch may be created automatically by a processor based on the three-dimensional model of the cardiovascular structure. In some such embodiments, the processor may create the three-dimensional model of the patch based on a known normal cardiovascular structure geometry.

According to exemplary embodiments described herein, a three-dimensional model for a patch to be applied to a cardiovascular structure may be based on a database of patch materials. In some embodiments, the database may include a plurality of anisotropic materials and their corresponding stress-strain curves. The stress applied to the patch may change depending on the desired geometry of the three-dimensional patch. Accordingly, in some embodiments a method of reconstructing a cardiovascular structure may include selecting a material from a plurality of materials based at least in part on the stress strain curves of the material. In some embodiments, the stress strain curves may be obtained by applying biaxial testing to a plurality of materials and storing the results in a database.

In some embodiments, a method of reconstructing a cardiovascular structure includes flattening a three-dimensional model of a patch into a two-dimensional plan. In some embodiments, the method may include providing the three-dimensional model of the patch. In some embodiments, the method may include receiving the three-dimensional model of the patch as the result of a method as described above. In some embodiments, the three-dimensional model of the patch may be flattened such that the patch may be constructed out of a planar anisotropic material. That is, the planar material may be employed to create a pre-curved three-dimensional structure by applying tension to the planar material in certain areas and sewing portions of the material together in targeted regions. In some embodiments, the three-dimensional model of the patch may include an outermost perimeter. In some embodiments, flattening the three-dimensional model of the patch includes identifying one or more cutouts formed in the patch that may be closed to introduce curvature along a major and/or minor axis of the patch. In some embodiments, the one or more cutouts may include notches in the patch extending inward from the outermost perimeter. The notches may be triangular in some embodiments. In some embodiments, each of the one or more notches creates a discontinuity in the outermost perimeter. That is, the one or more notches create an angular break in the otherwise continuous outer perimeter of the patch. In some such embodiments, the one or more notches may include a first edge and an opposing second edge. In some embodiments, the method may include projecting the two-dimensional plan of the patch onto an anisotropic material. The method may include cutting the anisotropic material along a border formed by the outermost perimeter and the one or more notches.

In some embodiments, a method of forming a patch for a cardiovascular structure may include projecting a two-dimensional plan of the patch onto at least one layer of an anisotropic material. In some embodiments, the two-dimensional plan is a flattened three-dimensional model of the patch. In some cases, the two-dimensional plan may be created according to the exemplary methods described above. The two-dimensional plan of the patch may include an outermost perimeter and one or more cutouts (e.g., notches) in the patch. In some embodiments, the one or more notches may extend inward from the outermost perimeter, where each of the one or more notches creates a discontinuity in the outermost perimeter. That is, the one or more notches may form an angular break in the otherwise continuous outermost perimeter of the patch. In some embodiments, the method may include cutting the at least one layer of the anisotropic material along a border formed by the outermost perimeter and one or more notches. A user may cut the anisotropic material according to the projection of the two-dimensional plan on a horizontal surface. In some embodiments, the projector may be a laser projector. Of course, in other embodiments, a template, another projector type, and/or other implements may be employed to assist in cutting the anisotropic material, as the present disclosure is not so limited. In some embodiments, the method may further include sewing, or otherwise joining, a first edge of each of the one or more cutouts to a second opposing edge of the one or more cutouts. The sewing or may apply tension to the patch and transform the planar anisotropic material into a pre-curved structure. In some embodiments, sewing the first edge to the second opposing edge of each of one or more cutouts includes removing the discontinuities in the outermost perimeter. In some embodiments, once the patch has the desired curvature, the pre-curved patch may be installed onto the cardiovascular structure (e.g., with sutures, adhesives, etc.) to reconstruct the cardiovascular structure into the desired geometry.

While in some embodiments sewing is employed to form a patch, in other embodiment other joining processes may be employed to form the patch, as the present disclosure is not so limited. For example, in some embodiments, an adhesive may be employed to join a first edge of a notch with a second edge of a notch. In some embodiments, a combination of sutures and adhesives may be employed to join a first edge of a notch with a second edge of a notch. In some alternative embodiments, a patch may be printed with a three-dimensional printer, such that no cutouts are employed.

While in some embodiments a projector may be employed to project a two-dimensional plan on a horizontal surface, in other embodiments a physical template may be employed. In some embodiments, one or more templates may be formed of stainless steel or another sterile material that correspond to a general two-dimensional patch shape for a cardiovascular structure. In some embodiments, a user (e.g., a surgeon) may be directed to select a template from a plurality of templates based on a patient's unique cardiovascular structure, physical characteristics (e.g., age, size, etc.), and/or based on genus or species of cardiovascular structural deformities. According to some such embodiments, the template may be used to cut out a patch from an anisotropic material, using the selected template border as a guide. The inventors have appreciated that use of a template tied to a type of cardiovascular structural deformities, even if not patient specific, may result in more consistent positive surgical outcomes.

According to the exemplary embodiments described herein, the methods described may be performed, in some embodiments, in whole or in part by one or more processors configured to executed computer-readable instructions stored in non-transitory memory. In some embodiments, the steps recited in methods described herein may be embodied as computer-executable modules in a software package stored on non-transitory memory and utilized by one or more users. The method may be broken into a plurality of modules to accomplish the various processes described herein that may be implemented either together and/or separately as the disclosure is not limited in this manner. Generally, program modules include routines, programs, objects, components, data structures, etc. that perform particular tasks or implement particular abstract data types. Typically, the functionality of the program modules may be combined or distributed as desired in various embodiments. Some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

In some embodiments, a patch for a cardiovascular structure may include at least one layer of anisotropic material including an outermost perimeter. In some embodiments, the anisotropic material may include one selected from the group of human cardiovascular homograft and autologous pericardium. Of course, any material may be employed, including bovine pericardium or expanded polytetrafluoroethylene, as the present disclosure is not so limited. In some embodiments, the patch may include one or more cutouts formed int eh anisotropic material. In some embodiments, the one or more cutouts may include one or more notches extending inward from the outermost perimeter. As discussed with reference to exemplary embodiments herein, the one or more notches may create an angular break in the outermost perimeter. In some embodiments, each of the one or more cutouts includes a first edge and a second opposing edge. The first edge and second edge may be configured to be joined together to apply curvature to the patch. In particular, the first edge and second edge may be configured to be joined together to provide a desired curvature for reconstruction of a cardiovascular structure (e.g., an aortic transverse arch). That is, in some embodiments, the one or more cutouts of the patch are sized and shaped such that the at least one layer has a curvature corresponding to the cardiovascular structure. In some embodiments, the patch further includes one or more sutures configured to secure the first edge to the second opposing edge. Of course, in other embodiments other suitable fasteners may be employed, including adhesives, as the present disclosure is not so limited. In some embodiments, joining the edges of the cutouts together may remove any discontinuities in the outermost perimeter, such that the patch has a continuous outmost perimeter free of angular breaks. In some embodiments, joining the edges of the cutouts together may remove any openings formed in the patch area, such that the patch has a continuous area free of holes. Of course, in other embodiments a patch may have an outmost perimeter with angular breaks or an area wile holes formed therein, as the present disclosure is not so limited.

While some exemplary embodiments described herein are discussed with reference to an aortic arch, it should be appreciated that the exemplary methods and systems of the present application may be employed for reconstruction of a wide variety of cardiovascular structures, including, but not limited to systemic arterial, pulmonary arterial, pulmonary venous, intracardiac structures, and systemic venous structures. Additionally, exemplary embodiments described herein may be applicable to non-cardiovascular structures, including, but not limited to, the gastrointestinal tract and airways. In one embodiment, this prospective patch design workflow can be applied to intracardiac baffles and patches to reconstruct complex heart disease. Such an embodiment may include patch design for: atrial septal defect closure, atrial baffle including atrial switch baffle for full or partial atrial switch (hemi Mustard or Mustard or derivations thereof), baffle of inferior vena cava and/or hepatic veins to right atrium, baffle of pulmonary veins to left atrium, patch closure of ventricular septal defect(s), baffle of left ventricle to the aorta, baffle of the right ventricle to the pulmonary artery, baffle of the left ventricle to the pulmonary, septation of a complete AV canal with closure of the ventricular septal defect and baffle of the left ventricle to the aorta, or repair of peripheral vessels including patch enlargement or repair of descending aorta and/or abdominal aorta, iliac vessels, femoral vessels or arterial or venous vessels of the extremities.

In another embodiment, the exemplary methods and patches of the present application can be developed for generalized anomalies for and then be utilized as an improvement over the current art. In one embodiment, a patch design for aortic arch augmentation for coarctation and hypoplastic transverse arch may be developed and translated into the operation room in a template. These templates could be a physical template (e.g., sterile plastic, metal or other material manufactured with molding, machining, 3D printing or other process) or an optical template (laser projection onto sterile field). Multiple sizes of templates may be available to correlate with size of the patient and or size of the native structure (e.g., transverse arch). In one embodiment, for coarctation and hypoplastic aortic arch, multiple sizes of templates could exist for each range of weights of the child. The individual template could then be selected based on the underlying arch anatomy (e.g., transverse arch size, ascending aorta size). In another embodiment, an arch reconstruction template for hypoplastic left heart syndrome patients undergoing a Stage I (Norwood) operation may be provided in a range of sizes. The size for a particular patient may be selected based on the size of the native anatomy including but not limited to the ascending aorta, main pulmonary artery, transverse arch and descending aorta. In another embodiment, the patch templates could be pre-cut out of patch materials based on pre-designed patches according to child size or anatomic specifications. In another embodiment, the pre-cut patches could have markings on the patch or additional template included with the patch to modify the patch based on a particular patient characteristic (e.g., transverse arch size). A patch for arch reconstruction in a Stage I could come pre-cut for a 3.0-3.5 kg neonate size with additional patch markings or included template that demonstrated trim areas for anatomic variations such as larger transverse arch or ascending aorta.

In addition to the above, the inventors have appreciated that reconstruction of an aortic arch to normal size and shape has several challenges that contribute to the variability of arch geometry after reconstruction that in turns contributes to the short-term and long-term risks of reintervention and poor cardiovascular health.

The inventors have recognized a first challenge is the marked impact of residual narrowing or dilation of the arch after reconstruction. It is desirable that the reconstructed aorta have a natural shape and taper to the descending aorta. Traditionally, surgeons have been so concerned with leaving residual narrowing, the default has been to make patches that err on the size of too large diameter by including excessive patch material. Patients with mild or moderate residual narrowing have residual afterload on the ventricle. However, similarly aortas with arch dilation that transition to a much smaller descending aorta have high impedance even without a significant measurable pressure drop. As the pulse wave travels through the dilated arch, then meets the smaller descending aorta, some of the energy is reflected back toward the ascending aorta. This reflected energy creates backward compression waves that increases the afterload on the ventricle. Over time, this pathologic, elevated impedance can have a lasting and detrimental impact on ventricular hypertrophy and ventricular function. The inventors have appreciated that due to these backward compression waves from unnatural diameter transitions in the aorta, the relative aortic caliber throughout the arch, and transition into the descending aorta, is the major determinant of afterload on the ventricle. Thus, the methods and systems according to exemplary embodiments described herein may be employed to prospectively design aortic arch reconstruction patches for patient specific arch reconstructions to achieve a natural caliber transition and curvature.

The inventors have recognized a second challenge is the impact of the mechanical properties of native aorta and patch materials which are currently only grossly estimated and taken into account by the surgeon during the reconstruction. Currently, the surgeon must reconstruct the aorta in a non-physiological, unpressurized state with the goal of it having a desired shape and caliber in the pressurized state. The inventors have appreciated the unique benefits of taking into account the mechanical properties (e.g., the elasticity, plastic deformation and residual stress after pressurizing) of the patch material and the native aorta when determining the size and shape of the patch. The anisotropic nature of pericardial and homograft patch materials and native aortas and their elasticity at physiologic loads may be taken into account in a quantitative manner to achieve a consistent desired arch caliber and shape in reconstruction. Thus, the methods and systems according to exemplary embodiments described herein may be employed to prospectively incorporate the mechanical properties of child aortas and clinical patch materials to engineer prospective patch designs that will have a known shape under physiologic conditions.

The inventors have recognized a third challenge is the multiplanar geometry involved in reconstruction. Coarctation treatment with arch hypoplasia is configured to (1) resect the area of coarctation and (2) reconstruct the aortic arch with a patch to achieve normal aortic arch shape and diameter. Patch materials employed for this reconstruction include pulmonary homograft or autologous pericardium. The inventors have appreciated that in order to recapitulate the natural shape of the aorta, a surgeon employing traditional methods faces the challenge of fitting a flat patch to the complex multi-planar curvature of the underside of the arch. During a conventional arch reconstruction process, the surgeon may visually estimate the patch size for the patient and iteratively adjust the patch size throughout the procedure in pursuit of a reconstructed arch with native aorta curvature and diameter throughout after the aorta is pressurized. As the aorta is pressurized, the patch is stretched in a very heterogeneous way resulting in some curvature of the patch but loss of elastic properties to match the compliance of the aorta. In the smallest aortas which require the largest patches, this lack of inherent patch curvature makes achieving a natural geometric shape even more challenging. Thus, the inventors have recognized the benefits of creating patient specific pre-designed patches with curvature to match the native aortic arch. The methods according to exemplary embodiments described herein may employ mechanical properties of native aortic tissue and patch materials and patient specific geometry to prospectively design aortic arch patches that will achieve natural shape and caliber under physiologic loads.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

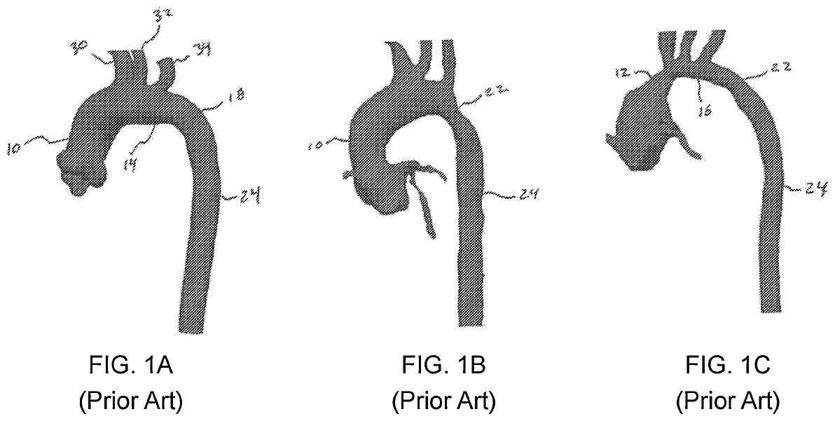

Shown in FIGS. 1A-1C is a representation of the variability of aorta anatomy that can exist in newborn children. Aortic arch hypoplasia and coarctation is a common clinical scenario in newborn children. There is an incredibly wide range of aortic pathology that can include the aortic root, ascending aorta, transverse arch, head vessels, coarctation in the area of the isthmus as well as the descending aorta. This can range in complexity from simple coarctation, which is a narrowing at the isthmus of the aorta to interrupted aortic arch with an aberrant subclavian artery. Consistent with the rest of congenital heart disease, even within standard diagnoses, there is wide variation and individual arch anatomy, which requires precise surgical reconstruction. Abnormalities in the aorta, aortic arch in these, the aorta is not limited to newborns. Older children can have arch abnormalities either congenital or acquired, which also need to be addressed. This can include arch hypoplasia, coarctation, or a range of anomalies. Children having an arch repaired earlier in childhood (including in newborns) can require intervention later in life if there is a residual arch disease or the arch did not grow appropriately with the child. The inventors have appreciated that the challenge surgically in all of these arch reconstructions is the individual variability of the arch anatomy requires the surgeon to adapt their reconstruction technique for each individual patient. This requires many intraoperative decisions about the reconstruction of the aorta, the patch size, and shape, the way the patch is sewn in place, and ideally, the mechanical properties of the aorta and the patch would be considered throughout this process.

FIG. 1A depicts a side view of an aortic arch is shown including ascending aorta 10, transverse arch 14 and descending aorta 24. There are multiple branches off the transverse arch including innominate artery 30, left carotid 32 and left subclavian artery 34. This arch has normal caliber of the ascending aorta, transverse arch, and descending aorta with no narrowing or abnormal curvature throughout the arch. FIG. 1B depicts a side view of an aorta with coarctation. This is a common aorta abnormality diagnosed as a neonate or infant. The ascending aorta 10 with transverse arch 14 and the descending aorta 24 with a coarctation 22 at the end of the distal arch of the transverse arch 14. This narrowing or coarctation is due to the development of ductal tissue in the isthmus area of the aorta just distal to the left subclavian artery. FIG. 1C depicts a side view of the aorta demonstrates a hypoplastic ascending aorta 12 with a hypoplastic transverse arch 16 in an area of coarctation 22 and a normal caliber descending aorta 24. Hypoplasia of the ascending aorta and transverse arch are commonly associated with coarctation and require more extensive reconstruction of the aorta than repair of coarctation alone. Often clinical repair of coarctation involves resection of the area of coarctation in the abnormal ductal tissue to exist and then re-anastomosis in the aorta in an oblique fashion to pull the descending aorta up into the distal aspect of the transverse arch. Although this technique can address mild hypoplasia in the distal transverse arch it cannot adequately address hypoplasia of the proximal transverse arch or the ascending aorta. Clinical repair of the aorta shown in FIG. 1C would usually include patch augmentation of the aorta including the ascending aorta, transverse arch, and extending that patch down to the descend aorta.

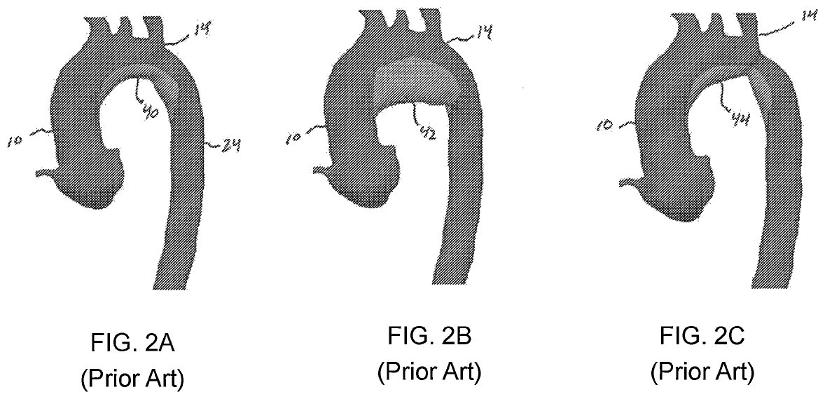

FIGS. 2A-2C depict some examples of an aortic arch after reconstruction according to conventional methods. Patch reconstruction of the aorta is commonly performed in the neonatal or infant after diagnosis of the arch abnormality. The techniques for arch reconstruction have evolved and improved and now commonly involve patch augmentation of the aorta. The traditional surgical approach involves supporting the body utilizing the heart lung machine in a manner such as that blood flow through the aorta can be stopped to allow for opening that aorta and a bloodless field for arch reconstruction. Typically, this involves inflow from the heart lung machine into the right innominate artery with support of the brain and the upper extremities during the period of arch reconstruction. This technique is known as regional cerebral perfusion. The lower body of the patient may be protected with moderate to deep hyperthermia or, alternatively, initial blood flow to the lower body may be introduced via a cannula in the descending aorta. The approach is to open up the arch in bloodless field and reconstruct it under zero pressure. The other head vessels are controlled with temporary snares or clips.

The conventional technique for sizing the patch and the shape of the patch both from a starting shape and a final shape varies widely among surgeons. The dominant variables above among the surgical approaches is how each individual surgeon was trained and how the institutional preferences have evolved for arch reconstruction. The practice patterns vary from cutting out a large general shaped patch that is then trimmed during the method of sewing the patch into the aorta versus precutting a patch to desired expected shape and size and then just sewing in place with no or little additional trimming. There are many variables that impact the geometric outcome of the aorta once it pressurized. The surgeons during the reconstruction typically try to take theses variables in to account with visual estimation and prior experience.

FIG. 2A depicts a side view of an ascending aorta 10 with transverse arch 14 and descending aorta 24 which has been reconstructed using patch 40 to treat arch hypoplasia and coarctation. In this particular embodiment, the patch extends down a portion of the ascending aorta across the transverse arch, across the isthmus area and down a portion the descending aorta. This aorta is shown in its pressurized state after arch reconstruction and represents a well-constructed aortic arch with normal caliber of the ascending aorta transverse arch and descending aorta. There is a natural shape to the arch and the head vessels. This represents an excellent surgical outcome after arch reconstruction and is the goal.

FIG. 2B depicts a side view of aortic arch following reconstruction of ascending aorta 10 which transverse arch 14 and the aorta has been reconstructed which a patch 42 it involves enlargement of the distal ascending aorta transverse arch and a portion of the descending aorta. The size and shape of the patch 42 has resulted in a pressurized aorta that has a transverse arch that is larger in diameter than is anatomically normal for this child. This a common geometric outcome following arch reconstruction given the approach of most surgeons to try to explicitly avoid residual arch hypoplasia but in doing so sometimes over sizing the patch resulting in an area of mild, moderate or profound transverse arch enlargement. Although this does not result in a physical obstruction of the aorta, it does increase the vascular impedance of the aorta in a very significant way. As the blood flow in the transverse arch transitions to the descending aorta, it has to accelerate, and this results in reflection of velocity waves at the transition to the descending aorta back through the transverse arch and into the ascending aorta which in turn increases the impedance of the left ventricle. In addition to increase in the impedance and afterload for the left ventricle or systemic ventricle and increasing the workload for that ventricle over the child's entire life, there are other implications of dilated transverse arch. This includes compression of local structures including the left pulmonary artery and the airway. Although it would be ideal if these dilated transverse arches would remodel over time as the children grew that in fact is typically not this scenario and they may remain a clinical problem for these children throughout their life.

FIG. 2C depicts a side view in an aorta with an ascending aorta 10 transverse arch 14 that has been reconstructed with a patch 44 to correct arch hypoplasia and coarctation. The patch 44 extends across the ascending aorta to transverse arch down a portion of the descending aorta. In this embodiment, the transverse arch 14 has an area of residual hypoplasia or narrowing at its distal aspect due to the shape of the patch 44 that was used for reconstruction. This is an example of the common clinical scenario where the patch had inadequate width of a portion and the result in the caliber of the aorta was smaller than desired. This residual narrowing results in increased afterload for the left ventricle and increase workload. Similar to the dilated transverse arch these areas of narrowing typically do not resolve on their own over time as the child grows but either negatively impacts the ventricle and the proximal aorta or require a re-intervention.

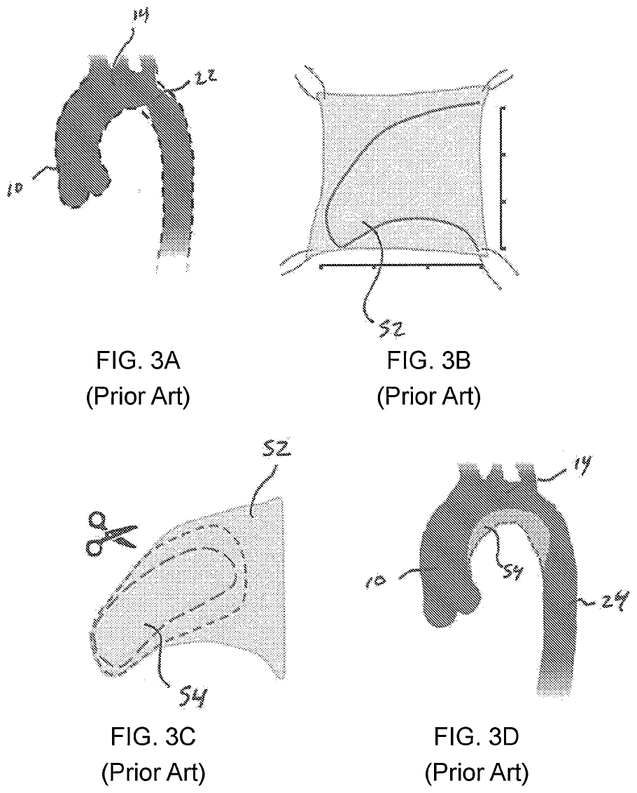

FIGS. 3A-3D depict a conventional aortic arch reconstruction workflow. FIG. 3A depicts a side view of an ascending aorta 10 with arch hypoplasia in an arch 14 and coarctation 22. FIG. 3B depicts a patch material 52 selected for arch reconstruction. As shown in FIG. 3B, a surgeon may trace a general outline for the patch based on the observed aorta. FIG. 3C depicts a method of cutting the patch material 52 into a patch 54. The patch material is sized and cut into initial shape by the surgeon, and further modifications shown in the dashed lines may be made by the surgeon during implantation based upon visual estimate. Once the patch 54 is sized to the surgeon's preference, it is attached to the aortic arch 14. The patch 54 may be attached to the aorta with sutures, and the surgeon may form the trimmed patch to the desired curvature if possible. FIG. 3D depicts a side view of aortic arch after patch reconstruction with the patch 54 installed in the arch 14.

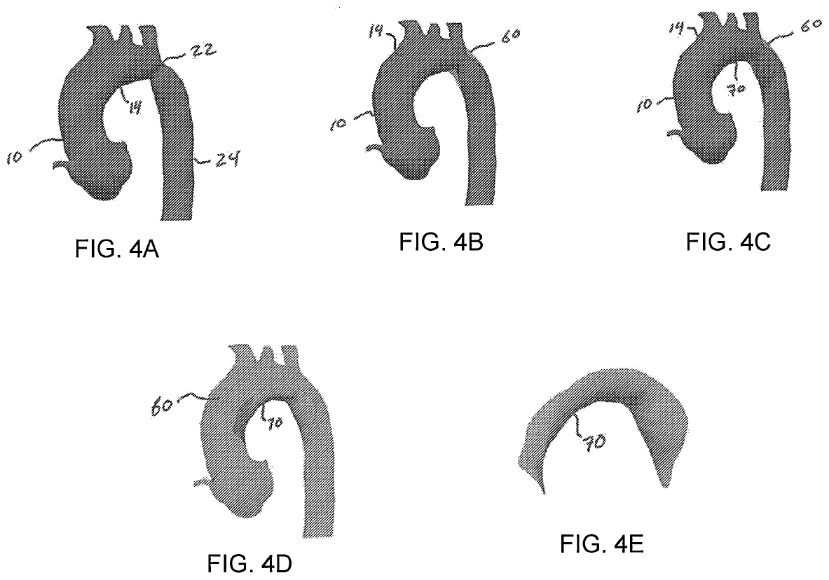

FIGS. 4A-4E depicts an exemplary method for developing an aortic arch patch (e.g., a patch for a cardiovascular structure) to provide a planned geometric outcome. The method shown in FIGS. 4A-4E may result in the determination of a three-dimensional shape of the patch to achieve the desired reconstruction. FIG. 4A depicts a side view of an ascending aorta 10 with arch hypoplasia in an arch 14 and coarctation 22. The aorta includes a normal caliber descending aorta 24. In some embodiments, the method may include imaging the aorta or produce one or more images of the aorta. The one or more images may in turn be employed to generate a three-dimensional model like the depiction shown in FIG. 4A. The three-dimensional model of the aorta may then be manipulated by a doctor to achieve a desired aortic geometry. The developed workflow utilizes no normal values for patients based upon their physical size, typically, body surface area. Using this data, the expected size of the ascending aorta, transverse arch, and isthmus area can be precisely planned as part of the reconstruction.

FIG. 4B depicts a subsequent step in the method of FIGS. 4A-4E, showing an adjustment 60 made to eliminate the coarctation shown in FIG. 4A. In particular, the model has been adjusted to show the changes in the geometry desired for reconstruction. In some embodiments, the adjustment shown in FIG. 4B may be input manually by a doctor (e.g., at a user interface). In some embodiments, the adjustment 60 may be generated automatically (e.g., by a processor) based on a stored known geometry (e.g., a normal geometry). The three-dimensional model of the patient's aorta can be derived several ways. Clinical imaging such as a CT scan or MRI can be utilized along with segmentation software to create a patient-specific three-dimensional model of the arch for this workflow planning. Alternatively, an echocardiogram may be utilized to dimension a 3-D model to achieve a patient-specific model prior to utilizing this workflow.

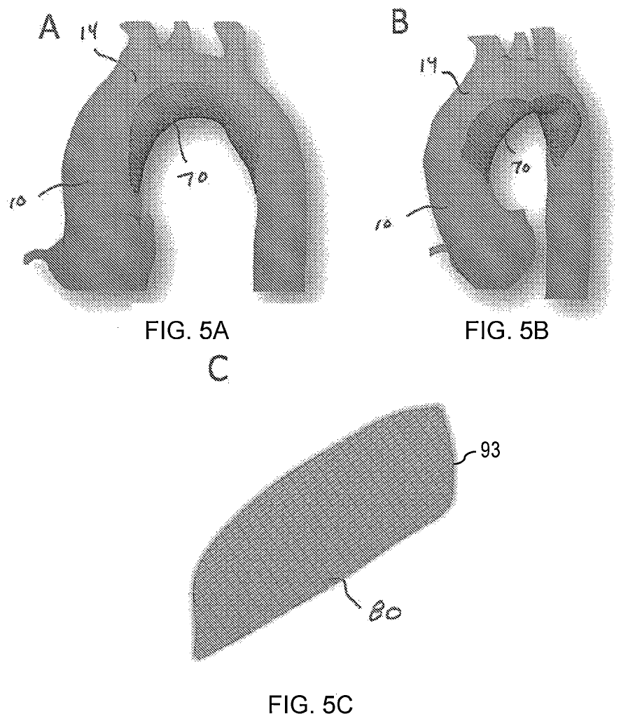

FIGS. 4C-4D depict a subsequent steps in the method of FIGS. 4A-4E, showing a three-dimensional patch model 70 configured to effect the adjustment determined in the step of FIG. 5B. As shown in FIG. 4C, the patch model, if implemented, is configured to eliminate the coarctation shown in FIG. 4A. The patch model 70 may represent an approximate ideal shape of a patch to achieve the desired reconstruction of the aorta. The patch model may be based at least in part on the patch material characteristics. Due to the presence of anisotropy for the materials used for aortic arch reconstruction, if the patch material mechanical properties were not considered or the patch was not rotated in a way to align the patch in such that the amount of stretch in the patch when pressurized can be accounted for, there could be a tremendous difference in the expected and resulted patch size once it is pressurized. Accordingly, in some embodiments the method includes first determining a patch size for the pressurized aorta. Second, the expected patch material properties may then be incorporated to adapt the patch to the expected unpressurized patch size and shape. Consideration of these mechanical properties allows the patch to be defined in terms of its unpressurized form with an expected increase in the patch size in both the major and minor axes once the patch is incorporated in the aorta and completely pressurized. Such an arrangement may ensure that the patch achieves the desired reconstruction once installed.

FIG. 4D depicts an isometric view of this three-dimensional model of a patch shunt. The size and the shape of the patch is configured for the patient's individual unique geometry based upon their aortic pathology. Although the patch may be similar for patients with similar general diagnoses such as coarctation with hypoplastic transverse arch, every patient is different. More complex arch pathology such as interrupted aortic arch or staged I single ventricle palliation with arch augmentation and Damus-Kaye-Stansel procedure would have a very different size and shaped patch. Accordingly, the method of FIGS. 4A-4E can be applied across a very broad range of arch pathology.

FIG. 4E depicts a final output of the method of FIGS. 4A-4E, which is a three-dimensional patch model 70 that is configured to achieve the reconstruction for the patient specific aorta 10. The patch model shown in FIG. 4E depicts the loaded or pressurized state which is different from the state in which the patch is cut and sewn in place. To integrate the product shown in FIG. 4E in the operating room, the three-dimensional shape for these patches may be converted to the flat patches that are clinically available in the operating room, as will be discussed further with reference to FIGS. 5A-5C.

FIGS. 5A-5C depict an exemplary method for developing an aortic arch patch (e.g., a patch for a cardiovascular structure) to provide a planned geometric outcome for the aorta. In particular, FIGS. 5A-5C depict a method of developing the patch for manufacturing by flattening of the aortic arch patch. FIG. 5A depicts a side view of aorta 10 with planned arch 14 reconstruction with patch model 70. FIG. 5B depicts an isometric view of the three-dimensional patch model 70 for aortic arch reconstruction as discussed above with reference to FIGS. 4A-4E. The patch may be ultimately formed of a planar material. Accordingly, as shown in FIG. 5C, the method includes flattening the patch such that the patch may be initially constructed in two-dimensions prior to being assembled in three-dimensions. Accordingly, as shown in FIG. 5C, the patch model 70 is converted into a two-dimensional plan 80 of the patch. In some embodiments, the patch model 70 and selected patch material may be such that the three-dimensional shape may be achieved with stretching of the patch. Accordingly, as shown in FIG. 5C, the patch may include a continuous outermost perimeter 93. In other embodiments as will be discussed herein, curvature may be introduced into the ultimately assembled patch by identifying one or more notches in the patch that can be closed to add curvature to the patch.

As shown in FIG. 5C the patch plan 80 is a flattened version of the three-dimensional patch plan. It is important the curvature of the 3-dimensional patch and the amount of curvature that the patch material can achieve is considered. If the curvature of the patch exceeds the mechanical properties of the patch due to achieve that curvature, more than one patch may be employed to achieve the desired target reconstruction geometry. This may include separate patches for the descending aorta, transverse arch segment, and ascending aorta segment. In other complex diseases, it could mean a separate patch for the proximal portion of the ascending or if enlargement of the aortic root was required as part of the reconstruction. The patch plan 80 may be shaped into sterile patch materials in the operating room. In some embodiments, the dimensions of the patch plan 80 may be manually transferred onto sterile patch material utilizing sterile rulers for measuring. In another embodiment the three-dimensional patch model can be transferred to a sterilized material as a 3-D printed shape, molded shape, or other fabrication methods to create a serializable patient-specific shape for the patch. In another embodiment, a laser system may be employed capable of transmitting engineering design files such as these shape files onto a surface that could be utilized in the operating room to project the size and the shape of the desired patch onto the sterile patch material. That can be traced out and the patch material cut to the appropriate size and shape utilizing the same sterile workflow of the normal patch materials.

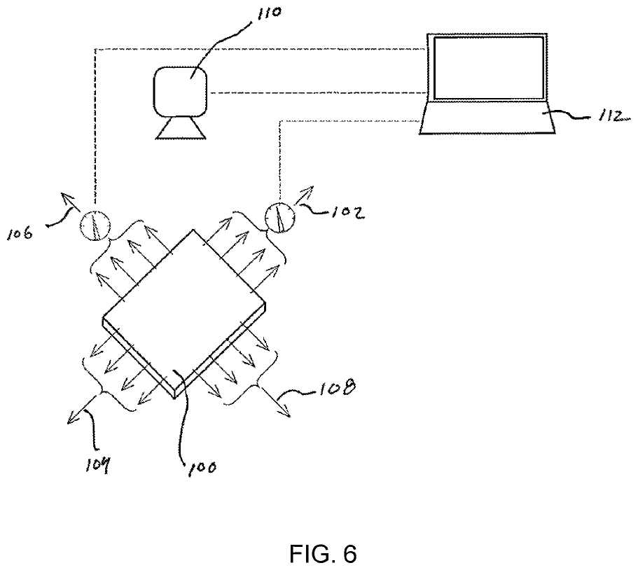

FIG. 6 depicts a schematic of a biaxial testing of anisotropic patch material. Such testing may contribute data to a database that may be employed to determine an appropriate three-dimensional model (and ultimately a two-dimensional plan) of a patch in an unstressed state. That is, a patch material used for aortic arch repair are anisotropic and the degree of anisotropy is profound enough that the direction of the material at the time of implantation should be considered to achieve the most predictable geometric and mechanical outcome for the arch reconstruction. A plurality of patch materials for arch reconstruction may be mechanically tested or marked with the axis of the most stretch or the corresponding orthogonal access to less stretch. These mechanical properties may be determined for each patch material by mechanically testing the patch material. This is done with an apparatus approach known as biaxial testing. As shown in FIG. 6, a patch material 100 is placed on an apparatus where force can be pulled into orthogonal directions along the major and minor axes of deformation of the material. For biaxial testing, the major and minor stretch axes are determined beforehand. Then the patch is loaded accordingly in such that in one axis 102 and 104 a force is applied on the 102 axis. Simultaneous a force is applied on orthogonal axes 106 and 108 with a force being applied on one end as shown in 106 in the diagram. The positional change of the patch is recorded by a camera 110 and the camera and force information are fed into a computer 112. This information can be utilized to calculate the mechanical properties including the stress and strain curves for each separate access of the material.

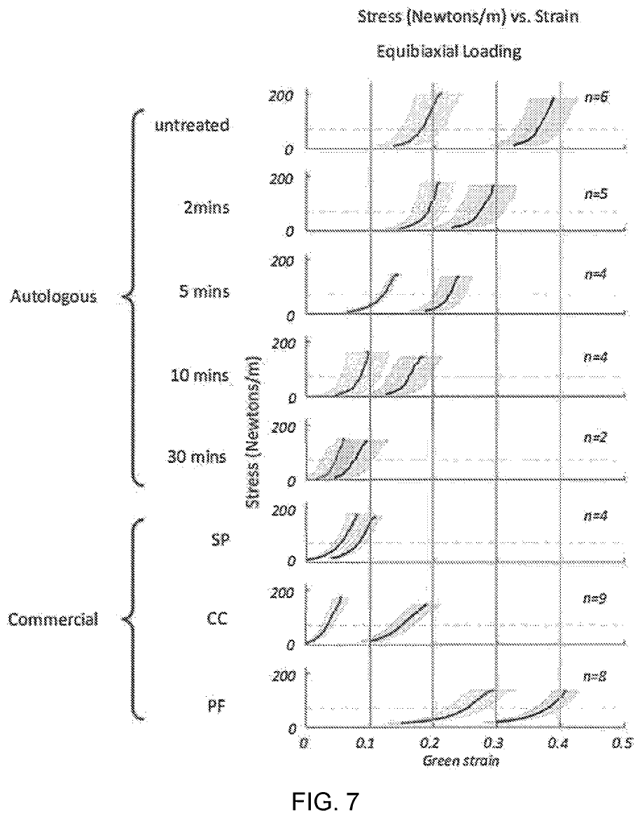

FIG. 7 depicts graphs showing the stress strain curves for the mechanical properties of pericardial patches. These graphs highlight the different mechanical properties for autologous pericardium that is treated in glutaraldehyde for varying amounts of time from untreated to up to treated for 30 minutes. It also highlights three commercial bovine pericardial patches and their differing mechanical properties. As is evident in the mechanical graph the properties including the degree of elasticity of the materials along their two different axes and vary widely among these pericardial patches. The testing described with reference to FIG. 6 may be employed to understand both the direction and degree of deformation that these patches will undergo when used to augment the aorta. The expected pressure of the aorta is important to take into consideration as it will determine the expected stretch deformation of the patch under those leading conditions. For example, a neonate undergoing arch reconstruction with a mean blood pressure between 40-50 mmHg or in an adult or older child may have a mean pressure between 70-90 mmHg.

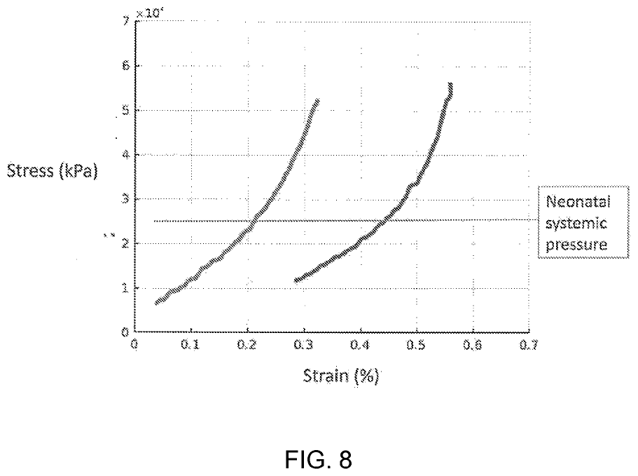

FIG. 8 depicts a stress strain curve shown for a pulmonary homograft patch. Highlighted in this stress strain curve is the level of corresponding neonatal systemic pressure. The pulmonary homograft patch can stretch in the range of 40% in one direction and 20% in an alternative direction. The exact stress strain measurements vary for different homograft patches. However, the graph shown in FIG. 8 is representative and demonstrates the degree of anisotropy of these patches and the importance of incorporating the patch material mechanical properties including the direction of anisotropy into the planning of patch reconstruction of a cardiovascular structure like an aortic arch. Given the anisotropic nature of these patch materials and the typically orthogonal relationship between the major and minor axis of the pulmonary homograft tissues, the patch material's principal material direction may be determined. The principal material direction is the direction of the greatest stretch or a strain for a given load. The material's principal material direction may be employed along with the anisotropic material test data for the formation of a three-dimensional patch model. Not knowing the principal material direction could lead to significant variation in the final shape of the patch given how fundamentally different the mechanical properties, particularly the strain under physiologic loads, can be.

In some embodiments, a database for both the infant aorta and pulmonary homograft data may be formed. Such information may allow the used finite element analysis (FEA) or other tools to calculate the expected strain for the patch continuously across the entire surface of the patch. In some embodiments, the maximum allowed strain for the patch materials may be defined in the database. The database may enable a preset of the maximum amount of strain or deformation of an area of the patch at physiologic loads. Such a preset allows the patch to be designed considering it will be sewn in non-pressurized conditions and then inflated with a physiologic pressure. Given the elasticity of these patch materials (particularly pulmonary homograft) it may be desirable to design the patch such that the post pressurized shape does not stretch the patch to its elastic limit by curving in so many directions to a tremendous degree. Traditional clinical arch reconstruction approaches rely on the elasticity of the patch to create the curvature in the reconstructed arch once the vessel is pressurized, creating very differential mechanical properties of the patch after implantation. As discussed previously, this creates variations in compliance that may be a significant component of complex impedance, which may be higher in these current reconstructed arches even if the geometry is relatively acceptable.

As they are both elastic and anisotropic, mechanical testing of native infant aorta tissue and pulmonary artery homograft tissues may exhibit a relatively wide range (>10% variance) of mechanical behaviors even when normalized to thickness. This may make application of library data of mechanical properties less accurate when describing an individual aorta or in particular a cryopreserved patch. Additionally, as these patches are typically 2-3 cm in length when used clinically, there is a possibility of variation of the mechanical properties along the length of a patch. If a wide range of mechanical properties is identified, individual testing of each patch may be employed.

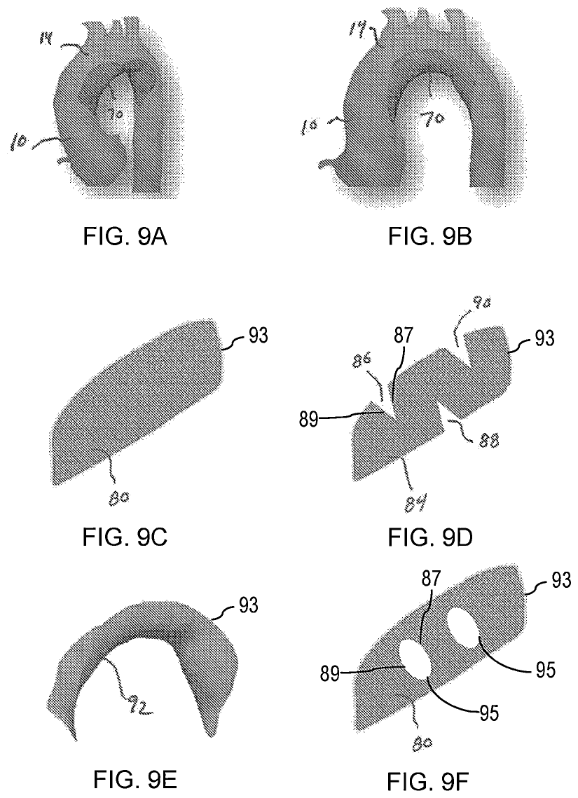

FIGS. 9A-9F depict an exemplary method for developing an aortic arch patch (e.g., a patch for a cardiovascular structure) to provide a planned geometric outcome for an aorta 10. In particular, FIGS. 9A-9F depict a method of developing the patch having pre-curvature to achieve the desired outcome when installed on the aorta. FIG. 9A depicts an ascending aorta transverse arch 14 is shown with a three-dimensional patch model 70 in place to achieve a desired geometry. FIG. 9B depicts the ascending aorta transverse arch 14 and patch model 70 are shown in a side view demonstrating the three-dimensional curvature of the planned patch for arch reconstruction.

As discussed previously in reference to FIGS. 5A-5C, the patch geometry from the patch model 70 can be flattened to translate the shape and size of the patch to a two-dimensional patch plan 80, as shown in FIG. 9C. In some embodiments, the method may include identifying areas where the curvature would exceed the expected max of the curvature of the patch materials. In cases where the curvature exceeds the expected max, material can be removed to reduce pre-curvature of the patch. In some embodiments, the method may include identifying one or more areas to remove from the patch to achieve the desired curvature. In some embodiments, the one or more areas may be notches (e.g., triangular notches, as shown in FIG. 9D.

FIG. 9D shows a modified two-dimensional patch plan 84. That is, the patch plan 80 of FIG. 9C has been modified by removing areas of the patch material 86, 88, and 90. In particular, as shown in FIG. 9D, the areas are configured as triangular notches. These areas of the patch are configured to have material removed and then the patch is sewn back together in those areas to induce curvature of the patch. As shown in FIG. 9D, the removed areas introduce discontinuities in the otherwise continuous outermost perimeter 93. That is, the removed areas create angular edges 87, 89 extending from the outermost perimeter in an inward direction relative to the patch plan. In some embodiments, each notch of the plan includes a first edge 87 and a second opposing edge 89 which are configured to be joined (e.g., with sutures, adhesives, etc.). In some embodiments, when attached to one another, the first edge and second edge may introduce tension and three-dimensional curvature into the manufactured patch. Accordingly, by removing areas of the patch plan 80, a pre-curved patch may be formed from a two-dimensional plan. In some embodiments, joining the first edge and second edge 89 of each notch may also eliminate the discontinuities in the outermost perimeter 93. The notches shown in FIG. 9D may introduce curvature along a minor axis of the patch.

As shown in FIG. 9E, once the edges 87, 89 of the areas removed from the patch plan are joined, the patch may regain curvature. FIG. 9E depicts a curved patch 92 formed from the modified patch plan of FIG. 9D. The curved patch 92 is now ready for incorporation of the aorta 10 with the pre-curvature in place and the size and shape set to match the aorta's existing geometry to create a targeted reconstructed aorta size and shape. The amount of pre-curvature employed for each individual aorta will vary. The factors that will impact whether pre-curvature of the patch is employed will include overall curvature of the patch and the mechanical properties of the patch materials being used. In some very complex geometries with patches that have less deformation under pressure, substantial patch curvature may be induced by one or more areas of the patch being removed to minimize the risk of undesired patch deformation including kinking. In other geometries there may two or fewer areas where the patch cut and pre-curved. Finally, in some embodiments, there are many patients where no pre-curvature will be employed because of the orientation of the patch and the amount of deformation that the patch can undergo will be enough to accommodate the curvature desired for the aorta reconstruction.

In some embodiments, to form the patch of FIG. 9E, a projection of the modified patch plan 84 may be projected onto at least one layer of an anisotropic material. A surgeon may employ the projection to cut out the patch following a border formed by the outmost perimeter 93 and the notches of the patch plan. The surgeon may then sew or otherwise join together the edges 87, 89 of each notch to form the pre-curved patch 92 shown in FIG. 9E. With a patch design including curvature, the patch elasticity can be preserved to maintain more uniform elasticity throughout the patch under physiologic loads. This will allow the patch to function in concert with the native tissue including normal wave propagation and the avoidance of energy loss from varying compliance. This will be a stark improvement over the current approach where the surgeon just hopes to avoid residual arch obstruction.

FIG. 9F depicts an alternative two-dimensional patch plan 80 including cutouts 95. In contrast to the notches of FIG. 9D, the cutouts 95 shown in FIG. 9F are interior cutouts. Similar to the notches of FIG. 9D, each of the cutouts includes a first edge 87 and second opposing edge 89 which may be joined together (e.g., with sewing, adhesive, etc.). According to the embodiment of FIG. 9F, the cutouts may be shaped as an ellipse. When the first edge 87 and the second edge 89 are joined, tension may be applied to the patch along a major axis of the patch. In this manner, interior cutouts like those shown in FIG. 9F may be employed to introduce major axis curvature into a patch. In some embodiments, when the first edge and second edge are joined, the area of the patch may be made continuous without holes.

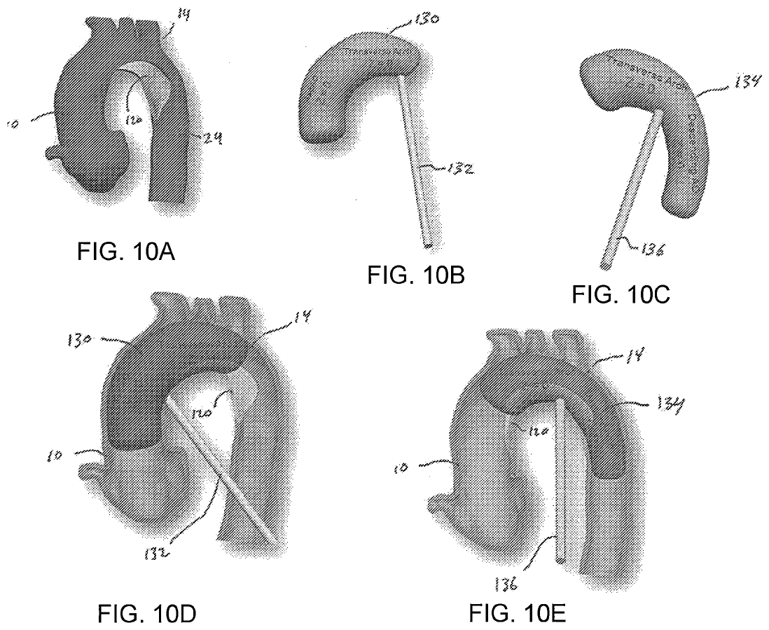

During the reconstruction of a cardiovascular structure such as an aorta, the patches sewn in place typically starting at the most distal aspect of the patch and proceeding more approximately. As the reconstructed structure (e.g., aorta) begins to take shape, currently surgeons eyeball the size and shape of the reconstructed structure. With perspective patch design of the size and shape of the patch for reconstruction, it may be a useful step to confirm that the resulting structure size and shape in the unpressurized state during patch implantation is as expected. Such an exemplary method is described with reference to FIGS. 10A-10E.

FIGS. 10A-10E depict one embodiment of a method of molding an aorta for validating the methods and systems described herein. Specifically, FIGS. 10A-10E depict use of a mold for intraoperative assessment and confirmation of patch side and shape. FIG. 10A depicts an isometric view of aorta 10 opened at an opening 120 on the underside of an arch 14. That is, FIG. 10A depicts an ascending aorta 10 with transverse arch 14 and descending aorta 24 where the underside the aortic arch has been opened at an opening 120 in preparation for a patch augmentation. In many cases the coarctation tissue of the aorta will have been excised and then the back wall the aorta reconstructed to achieve this opening on the underside of the aortic arch.

FIG. 10B depicts a mold 130 of an ascending internal portion of aorta of planned size and shape attached to a handle 132. The mold 130 matches the planned reconstructed size of the elements of the aorta including the ascending aorta 10 and arch 14 achieving a score of zero after reconstruction. During arch reconstruction this mold can be placed up in the arch to guide or reassure the surgeon on the appropriate size of the unpressurized aorta. FIG. 10C depicts a mold 134 of an arch of aorta of planned size and shape attached to a handle 136. The arch mold 134 and handle 136 can be utilized to guide the sizing of the arch and descending aorta. In another embodiment, the mold may incorporate aspects of the ascending aorta and transients arch and descending aorta or just one aspect of the aorta depending upon what is most desired by the surgeon. In another embodiment there may be no fixed handle but a keeper element such as a suture or string to retain that location of the mold. In some patients this may facilitate utilization of the mold inside the arch during reconstruction. In another embodiment the mold may be inflatable and may be able to be deflated for easy removal. In another embodiment the mold may not be solid but may be a collapsible or deformable material which may also ease placement and removal. In another embodiment, the mold may be adjustable wherein the surgeon could adjust the shape or size of the mold according to their preference intraoperatively.

FIG. 10D depicts a side view of aorta with the ascending internal sizing mold 130 in place. FIG. 10D demonstrates the positioning of the mold 130 and aorta during arch reconstruction or sizing for confirmation of the placement of the arch patch in its relative size. FIG. 10E depicts a side view of the aorta with the descending internal sizing mold 134 in place. The mold is 134 with handle 136 placed inside the cut edge of the under set of the opening 120 to confirm or demonstrate the size of the intended arch it is decompressed state during reconstruction. Thus, the molds 130, 134 may be employed to validate that a prepared patch will achieve the desired geometry of the aorta.

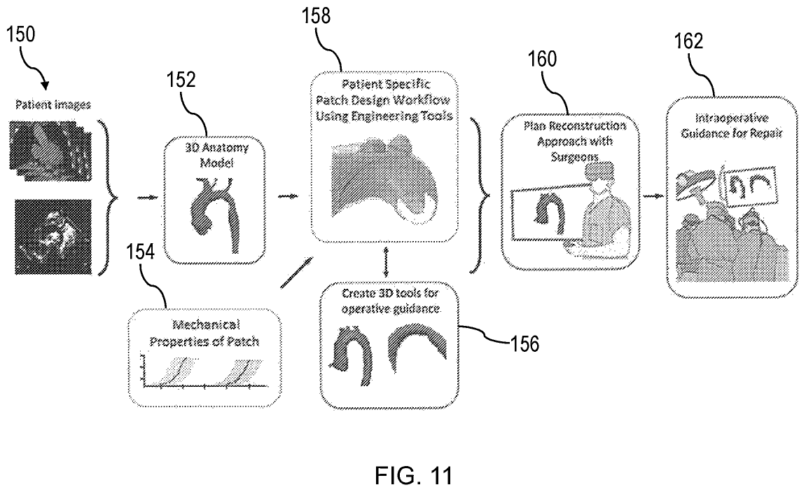

FIG. 11 depicts an overarching method for forming a patch for reconstruction of a cardiovascular structure like an aortic arch. At 150, patient images are provided. The patient images may be of the cardiovascular structure for reconstruction, and may be any suitable images. The patient images may include echo, CT, MRI, or other imaging modalities. In block 152, the images are converted into a 3D anatomy model of the cardiovascular structure. That is, the patient images are utilized to create a patient-specific three-dimensional anatomical model of the patient's arch and branches. In some embodiments, depending on the patient's anatomy the rest of the heart and surrounding vascular structures may be also created if interventions are desired in those areas as well. In block 154, the properties of a patch material, or a plurality of patch materials, is provided. Based upon the surgeon's preference or the most suitable material for arch reconstruction for an individual patient, the expected patch material is chosen. In block 156, three-dimensional molds may be created to assist in surgical reconstruction and installation of the ultimately produced patch. In block 158, a patient specific patch design is generated based on the anatomy model, patch material properties, and optionally the three-dimensional molds. The mechanical properties of that patch material either for the individual patch material or average data for that type of patch material are utilized to design a patient's specific patch utilizing the engineering tools outlined above. This includes the creation of the expected post-reconstruction geometry of the cardiovascular structure (e.g., aorta) and a calculation of the patch size and shape required to achieve that reconstruction in a pressurized cardiovascular structure. Utilizing the mechanical properties of the patch material the patch is scaled down to the expected patch size and shape where a decompressed or zero pressure patch material. This patch design may also be utilized to create the tools for operative planning and guidance in block 156. In block 160, the patch design and intraoperative tools are then reviewed by the surgeon performing the operation and the details of the surgical plan are determined. In block 162, the plans for patch size and shape and material may then be taken into the operating room where the arch geometry and plan reconstruction can be highlighted in several intraoperative visualization methods including but not limited to screens in the OR that may be adjacent to the operating table, three-dimensional glasses free 3D screens, images incorporated into eyewear including virtual or augmented reality and three-dimensional projections within eyewear. The size and shape of the patch may be translated to the sterile patch in the operating room as discussed above including but not limited to laser projection of the patch onto the sterile patch and or utilization of a prefabricated and sterilized template specific for the patch patient. The patch may be formed according to exemplary embodiments described herein. The surgical reconstruction using the three-dimensional molds and patient specific patch information may achieve the desired cardiovascular geometry consistently and reliably.

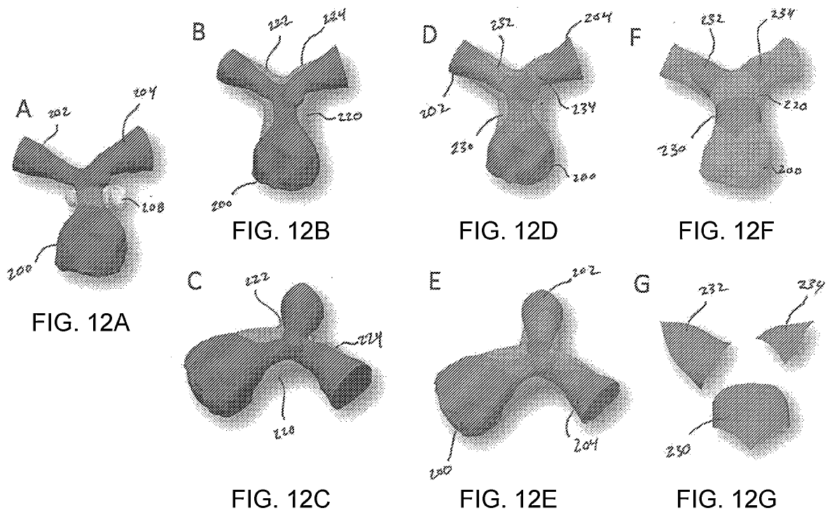

It should be noted that in addition to the prospective design of patches for arch reconstruction, the methods described herein may be adapted to prospectively design cardiovascular and non-cardiovascular reconstructions for a variety of use cases. In some embodiments, such methods may be utilized to prospectively design patches for pulmonary artery reconstruction. This is a common surgical scenario for patients with complex congenital heart disease either as part of a primary operation or subsequent operation. Somewhere to the arch the main pulmonary and branch pulmonary arteries are complex structures with taper and curvature and historically there have been challenges in achieving the exact desire geometry after patch reconstruction. A method of reconstructing such a cardiovascular structure is discussed further with reference to FIGS. 12A-12G.

FIGS. 12A-12G depict one embodiment of a method of reconstructing a cardiovascular structure other than an aorta. In particular, the method shown in FIGS. 12A-12G is directed to reconstruction of a main pulmonary artery 200 and branch pulmonary arteries 202, 204 with a PA band 208 in place, as shown in FIG. 12A. As shown in FIG. 12A, there is a restriction or pulmonary artery band around the distal pulmonary band 208 distal main pulmonary artery. A pulmonary band is often placed surgically to induce narrowing of the pulmonary artery, that is later removed surgically, and the pulmonary artery often requires reconstruction. Utilizing known normal values for main pulmonary and branch pulmonary sizes, the desired pulmonary artery reconstruction may be planned. The patient-specific anatomy can be acquired typically with CT scan or MRI imaging. A patient-specific three-dimensional model is created. The method may also include utilizing a normal anatomy and the calibers of the main pulmonary and branched pulmonary arteries and the three-dimensional model to create a prospective plan for the pulmonary artery reconstruction.

FIG. 12B depicts a top view of the modified main pulmonary artery 220 and branch pulmonary arteries 222, 224 after digital reconstruction. FIG. 12C depicts an isometric view of the main pulmonary artery and branch pulmonary arteries after digital reconstruction. Based on the desired digital reconstruction, a plan patch size may be calculated. FIG. 12D depicts a top view of the main pulmonary arteries and branch pulmonary arteries after digital reconstruction with computationally derived patches 230, 232, 234, derived according to exemplary embodiments described herein. That is, the main pulmonary artery 200 has branch arteries 202, 204. Pulmonary artery patch 230 is configured to reconstruct the main pulmonary artery and patches 232 and 234 are configured to reconstruct the proximal right and left pulmonary arteries. FIG. 12E depicts an isometric view of the main pulmonary artery and branch pulmonary arteries after digital reconstruction with computationally derived patches. FIG. 12F depicts a top view of the main pulmonary artery and branch pulmonary arteries after digital reconstruction with computationally derived patches. That is, FIG. 12F depicts the resulting three-dimensional patch augmented shape shown with the main pulmonary artery 200 reconstructed. The modified main pulmonary artery 220 is shown with patch 230, a right pulmonary patch 232, and a left pulmonary patch 234.

FIG. 12G depicts a top view of the three patches computationally derived for pulmonary artery reconstruction. The three patches are configured to achieve the desired pulmonary artery reconstruction including main pulmonary patch 230, right pulmonary patch 232, and left pulmonary patch 234. The augmentation of the pulmonary arteries can be accomplished with one or more patches depending on the clinical and geometrics scenario for the individual patient. The method allows for the adaptation of the patches to accommodate different options. Like the aortic arch patch workflow, the expected mechanical properties of the patch are considered, and patches may be accordingly downsized based upon the expected stretch of the patch under the physiologic properties of the pulmonary arteries. In some embodiments, one or more of the patches may be flattened such that the patches may be formed of a planar anisotropic material. As with previously discussed embodiment, pre-curvature of the patch can be achieved with removal of identified areas of the patch and then resewing, rejoining, or shaping the patch back together to induce the desired curvature in the patch.

### Examples

A clinical workflow has been developed that utilizes patient specific images to perform digital analogues of surgical procedures for preoperative planning and intraoperative guidance. This clinical workflow has achieved clinical impact by utilizing modeling and simulation for preoperative planning of patients with single ventricle heart disease. This workflow includes the creation of patient specific models from CT scan and MRI data sets. We combined patient specific models with the CATIA platform digital tools to realize complex operative reconstructions of cardiac anatomy including 3D curvatures and organic shapes and, in many cases, predict clinical results with computational fluid dynamics analysis. We have implemented this clinical workflow in over 100 patients in our heart center.

Building on the CATIA 3D modeling and design platform, arch reconstruction was performed on patient specific models where coarctation or arch hypoplasia exists. The challenge was to incorporate the mechanical properties of the patch material and the native aorta with the complex 3D curvature the patch will assume to accurately re-create the shape and caliber of the native aorta or of a normal-sized aorta for the child. A first step in this process was to determine the mechanical properties of both the native aorta and patch materials. Extensive testing of autologous pericardium unfixed and after various levels of fixation of glutaraldehyde was performed. Autologous pericardium has a distinct anisotropic behavior and the strain after fixation is dependent on the amount of glutaraldehyde fixation. Pulmonary homograft which is another patch material used for aortic arch reconstruction also has distinct anisotropic properties. As there was limited available data on these patches, a mechanical property library of these patch materials was built. The integration of anisotropic mechanical properties of patch material and the target 3D shape of the patch was an important technical hurdle that until recently has been a barrier to advancement for this effort. A composite tool was developed specifically for this purpose.

Utilizing the CATIA 3D design platform in this composite tool, reconstruction of both generic and patient specific hypoplastic aortic arches was accomplished. Combining usual operative approaches with these engineering tools has allowed us to create a workflow whereby which the aorta can be effectively digitally opened just as we would in the operating room and then reconstructed to a normal shape and curvature. The resulting patch shape to achieve this can be defined. The mechanical properties of the patch are also incorporated, specifically how much the patch will stretch to transform the patch from a flat but elastic material into a complex 3D shape. The composite software tool calculates the local strain or deformation across the entire patch. This gives a user the specific locations where the patch may or may not be able to curve adequately to achieve the desired shape of the design patch. Areas where more deformation is desired then the patch material will tolerate are identified in the composite design tool. These areas of the patch can be removed and with exact precision by design and the patch sewn back together to induce pre-curvature of the patch so the resulting shape of the aorta that is desired can be achieved.

Using methods described above according to exemplary embodiments herein, coarctation within a hypoplastic aortic arch has been digitally corrected and the appropriate patch design accomplished. In this example, given the input mechanical properties of pulmonary homograft, the patch material can adequately stretch during physiologic loading to accomplish the desired shape when cut appropriately. In this case, the software identified several areas they were near the maximal amount of strain or deformation capable by the patch but none of it required pre-curvature in this example. A much more complex patient specific aortic arch may include diffuse hypoplasia of the isthmus area, transverse arch, and even ascending aorta. Utilizing the composite tool software, the desired natural caliber and shape of the aorta was prescribed and then the patches were designed with calculation of the maximal strain continuously across the patches. In this case, utilization of three patches, one on the underside of the arch down the ascending aorta, and a second and third patch along the medial and lateral aspect of the ascending aorta achieved reconstruction of the aorta to completely normal caliber throughout its entire course, and was achievable within the maximal strain or deformation of the pulmonary homograft patch. Had a less elastic patch material such as autologous pericardium been utilized, pre-curvature of the patch would have been induced. Thus, these tools allow a user to reconstruct cardiovascular structures and make the operation as feasible and efficient as possible and still achieve a normal cardiovascular structure in shape.

The flattening or unfolding of complex patch shapes has also been achieved utilizing an FE flatten tool available within the CATIA platform. In an exemplary scenario, a complex single ventricle patient undergoing a Fontan revision needed a special design for a transition between 20 mm extra cardiac Fontan conduit and with 38 mm opening to the branch pulmonary arteries. A transition cuff was designed that had a complex 3D shape. The mechanical properties of the expected patch material were incorporated into the design. The shape of the cuff was optimized using computational fluid dynamics analysis with iterative improvement of the patch shape to achieve minimal energy loss and a good flow distribution to both lungs. Using these flattening tools, this complex 3D cuff shape was unfolded to the corresponding two-dimensional shape where the cuff could be created using a patch material in the operating room. The cuff was successfully reconstructed and the patient underwent the operation with an excellent clinical outcome. Postoperative MRI demonstrated excellent congruence between the planned and the actual reconstructed shape of the Fontan pathway.

Sterile transfer of patch designs to patch materials in the operating room may be performed with a laser projector system or via another suitable process. Translating the patch designs to sterile patch materials in the operating room to be used clinically is an important step towards translating this technology. In some cases, patch designs may be manufactured of stainless steel in a large variety of shapes (e.g., templates) to accommodate the range of patch designs applicable to neonates or other types of patients. In some embodiments, a more patient specific approach is to three-dimensional print patch designs in custom sheets under a clinical workflow that allows them to be sterilized and utilized on the OR table but not as an implant. Flexibility may be provided to a user via the utilization of a laser projector within the operating room that projects the patch design down onto a patch material directly. The patch material can then be marked and then cut out by a surgeon. Such laser projector systems are designed to work directly with 3D engineering design tools include the CATIA platform that our team is utilizing. In some embodiments, a portable custom cart may be used to mount the laser in the operating room to transfer design patterns to the patch materials. The shape and dimensions of the patch that is cut out will be dimensionally analyzed optically and compared the intended design.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A method of reconstructing a cardiovascular structure, the method comprising:
obtaining a three-dimensional model of the cardiovascular structure based on information regarding the cardiovascular structure; and
creating a three-dimensional model of a patch corresponding to the three-dimensional model of the cardiovascular structure, wherein the patch is configured to reconstruct the cardiovascular structure to a normal geometry.

2. The method of claim 1, further comprising characterizing the cardiovascular structure.

3. The method of claim 2, wherein characterizing the cardiovascular structure includes producing one or more images of the cardiovascular structure.

4. The method of claim 1, wherein obtaining a three-dimensional model of the cardiovascular structure includes producing the three-dimensional model based on the information regarding the cardiovascular structure.

5. The method of claim 1, further comprising flattening the three-dimensional model of the patch to a two-dimensional plan.

6. The method of claim 5, wherein the three-dimensional model of the patch includes an outermost perimeter, wherein flattening the three-dimensional model of the patch includes identifying one or more cutouts in the patch.

7. The method of claim 6, wherein the one or more cutouts include one or more notches extending inward from the outermost perimeter, wherein each of the one or more notches creates a discontinuity in the outermost perimeter.

8. The method of claim 7, wherein each of the one or more notches is triangular.

9. The method of claim 6, wherein the one or more cutouts are interior cutouts.

10. The method of claim 6, further comprising projecting the two-dimensional plan onto at least one layer of anisotropic material.

11. The method of claim 10, further comprising cutting the at least one layer of the anisotropic material along a border formed by the outermost perimeter and the one or more cutouts.

12. The method of claim 10, wherein projecting the two-dimensional plan includes projecting the two-dimensional plan onto a horizontal surface with a laser projector.

13. The method of claim 5, further comprising:
selecting a patch template corresponding to the two-dimensional plan from a plurality of templates; and
cutting at least one layer of an anisotropic material along a border formed by the patch template.

14. The method of claim 1, further comprising printing the three-dimensional model of the patch using an anisotropic material.

15. A non-transitory computer readable memory including processor executable instructions that when executed perform the method of claim 1.

16. A patch for a cardiovascular structure, comprising:
at least one layer of anisotropic material including an outermost perimeter; and
one or more cutouts formed in the at least one layer of anisotropic material.

17. The patch of claim 16, wherein the one or more cutouts are interior cutouts.

18. The patch of claim 16, wherein the one or more cutouts are one or more notches extending inward from the outermost perimeter, wherein each of the one or more notches creates a discontinuity in the outermost perimeter.

19. The patch of claim 16, wherein each of the one or more cutouts includes a first edge and a second opposing edge, wherein the first edge and the second edge of each notch are joined together.

20. The patch of claim 19, wherein the joined first edge and second edge of each of the one or more cutouts are configured to apply tension to the least one layer of anisotropic material to curve the at least one layer of anisotropic material.

21. The patch of claim 20, wherein the one or more cutouts are sized and shaped such that the at least one layer has a curvature corresponding to the cardiovascular structure.

22. The patch of claim 21, wherein the cardiovascular structure is an aortic transverse arch.

23. The patch of claim 19, wherein the one or more cutouts are one or more notches extending inward from the outermost perimeter, wherein each of the one or more notches creates a discontinuity in the outermost perimeter, and wherein the joined first edge and second edge of each of the one or more notches are configured to remove the discontinuities in the outermost perimeter.

24. The patch of claim 16, wherein the anisotropic material is one selected from the group of human cardiovascular homograft and autologous pericardium.

25. A method of forming a patch for a cardiovascular structure, the method comprising:
projecting a two-dimensional plan of the patch onto at least one layer of anisotropic material, wherein the two-dimensional plan is a flattened three-dimensional model of the patch, wherein the two-dimensional plan of the patch includes an outermost perimeter and one or more cutouts in the patch.

26. The method of claim 25, wherein the one or more cutouts are interior cutouts.

27. The method of claim 25, wherein the one or more cutouts are one or more notches extending inward from the outermost perimeter, wherein each of the one or more notches creates a discontinuity in the outermost perimeter.

28. The method of claim 25, further comprising cutting the at least one layer of the anisotropic material along a border formed by the outermost perimeter and one or more cutouts.

29. The method of claim 25, wherein projecting the two-dimensional plan includes projecting the two-dimensional plan onto a horizontal surface with a laser projector.

30. The method of claim 25, wherein each of the one or more cutouts includes a first edge and a second opposing edge, the method further comprising sewing the first edge to the second opposing edge of each of the one or more cutouts.

31. The method of claim 30, wherein sewing the first edge to the second opposing edge of each of the one or more cutouts includes applying tension to the least one layer of anisotropic material to curve the at least one layer of anisotropic material.

32. The method of claim 30, wherein the one or more cutouts are one or more notches extending inward from the outermost perimeter, wherein each of the one or more notches creates a discontinuity in the outermost perimeter, and wherein sewing the first edge to the second opposing edge of each of the one or more notches includes removing the discontinuities in the outermost perimeter.
